(19) United States
(12) Patent Application Publication
(10) Pub. No.: US 2017/0056574 A1
(43) Pub. Date: Mar. 2, 2017

(54) CATHETER FOR THE DIRECTIONAL CONVEYANCE OF A FLUID, PARTICULARLY A BODY FLUID

(71) Applicant: NovaPump GmbH, Jena (DE)

(72) Inventors: Joerg Pfeifer, Jena (DE); Ronald Reich, Jena (DE)

(21) Appl. No.: 15/257,647

(22) Filed: Sep. 6, 2016

Related U.S. Application Data
(63) Continuation of application No. PCT/DE2015/100081, filed on Mar. 2, 2015.

(30) Foreign Application Priority Data
Mar. 3, 2014 (DE) 10 2014 003 153.5

(51) Int. Cl. A61M 1/10; A61M 25/10; A61M 1/12; A61M 39/24

(52) U.S. Cl. CPC A61M 1/1098; A61M 39/24; A61M 25/10; A61M 1/1037; A61M 1/1072; A61M 1/122; A61M 1/125; A61M 2039/2473

(57) ABSTRACT

A catheter for the directional conveyance of a fluid, in particular a body fluid, is provided. The catheter includes a sleeve having an internal space and a frame. The sleeve has at least three openings and is configured at least in a region between the first opening and the second opening as a conduit for the fluid. A check valve is arranged at the second opening. The check valve includes a valve foil which is at least partially attached to the sleeve such that the second opening can be completely covered by the valve foil.

### CROSS REFERENCE TO RELATED APPLICATIONS

This application is a continuation application of international patent application PCT/DE2015/100081, filed Mar. 2, 2015, designating the United States and claiming priority from German application 10 2014 003 153.5, filed Mar. 3, 2014, and the entire content of both applications is incorporated herein by reference.

### FIELD OF THE INVENTION

The invention relates to a catheter for the directional conveyance of a (bodily) fluid, in particular a body fluid. The catheter includes a sleeve having an internal space and a frame, wherein the sleeve has at least three openings (and is fluid-tight with respect to the fluid being conveyed aside from these openings), and is designed at least in a region between the first opening and the second opening as a line for the fluid. A check valve is arranged at the second opening to make possible an exclusively unidirectional flow between the internal volume of the sleeve and the external space surrounding the sleeve.

### BACKGROUND OF THE INVENTION

In the context of the invention, the expression “fluid-tight” means an impermeability to atoms or molecules of the fluid in question, under the maximum conditions prevailing in this fluid in the living human body. If the catheter is, for example, designed for conveying blood, then “fluid-tight” means an impermeability at least up to a maximum human blood pressure.

A catheter of the type named above, having multiple check valves, is known in the prior art, for example from U.S. Pat. Nos. 8,932,246 and 8,409,128. The catheter is preferably used in cases of limited cardiac output to support the heart and the blood circulation. The catheter can also be used in cases of more advanced aortic regurgitation. The catheter serves the purpose of transporting the conveyed fluid from a first location to another location without significantly increasing the pressure of the fluid at the first location beyond a state determined by physiology by implementing the principle of a submersible pump and by combining it with the principle of a diaphragm pump due to the use of a balloon catheter. The direction of conveyance (flow direction) depends in this case on the orientation of the check valves. It thus enables, compared to the known intra-aortic balloon pump counterpulsation, a directional transport of the body fluid with less stress on the patient.

Such catheters can be called pump catheters for short. However, it is possible to use a separate drive, especially in the form of an adjustable displacement device, for example a balloon catheter of an intra-aortic balloon pump (IABP). The catheter is, in its basic form, simply a non-driven line catheter. The pump catheter can be implemented by pushing the displacement device following the placement of the catheter line, through the third opening into the internal space of the sleeve.

Therefore, it is possible and practical to implement the line catheter without a drive.

The complexity involved, and the negative impacts on the patient, in a minimally invasive insertion of a catheter into the body, for example via inguinal vessels, essentially depend on the size—and particularly the largest outer diameter—of the catheter.

### SUMMARY OF THE INVENTION

It is an object of the invention to provide a catheter of the type named above which has a smaller size.

The object is achieved by providing a catheter for directional conveyance of a fluid including: a sleeve having an internal space; a frame; at least a first opening, a second opening, and a third opening; said sleeve, at least in a region between said first opening and said second opening, being configured as a line for conveyance of the fluid; a check valve having a valve foil; said check valve being arranged at said second opening; and, said valve foil being at least partially attached to said sleeve so that said second opening is completely coverable by said valve foil.

According to an aspect of the invention, the check valve includes a (flexible) valve foil which is at least partially attached to the sleeve in such a manner that the second opening can be (reversibly) completely covered by the valve foil. For the purposes of the invention, the valve foil is a sheet of any material with a maximum thickness of 0.2 mm. Reversible means that the second opening can be alternately opened and covered by the valve foil.

The valve foil thus forms, with the sleeve, a foil valve which only allows unidirectional flow of the fluid through the same. In the direction of passage, fluid pressing against the valve foil can lift the valve foil of the second opening and thereby flow past the foil valve. In the direction which is blocked, the fluid presses the valve foil against the second opening, which is then covered as a result. Then the fluid cannot pass through the foil valve. Compared to the prior art, which describes mechanically rigid and thus space-intensive check valves, the foil valve according to an aspect of the invention is only a negligible presence due to the minimal thickness of the valve foil, such that the catheter when folded can have a lower greatest external diameter (hereinafter also referred to as total diameter) than the prior art. This is advantageous in particular in the field of cardiology because experience has shown that, when the overall diameter of cardiac catheters is reduced, it is necessary to exclude fewer patients of a particular patient population from a surgical operation due to their specific inner blood vessel diameter. In addition, the safety of the implantation and explantation can be improved by decreasing the overall diameter.

If there is no pressure difference between the internal space and the external space, the valve foil can either lie against the sleeve (due to a mechanical preload and/or due to its geometrical profile), or the valve foil can lie loosely in a position relative to the sleeve which is defined exclusively by the attachment thereof to the sleeve.

In general, the sleeve, particularly in the region of a radially symmetric or rotationally symmetric shell surface, can be shaped in such a manner that the second opening has an edge which is non-planar. The edge can be curved, for example, in three dimensions in the case of a cylindrical surface. Only foil valves according to an aspect of the invention enable, with minimal structural complexity, a fluid-tight seal of openings with edges curved in three dimensions.

The valve foil can be attached directly or indirectly to the sleeve. In the case of a direct attachment, the valve foil can be glued, welded, or clamped to the sleeve, for example along a line of attachment. The attachment along the attachment line can be continuous or discontinuous, for example in a dotted line. In the case of an indirect attachment, the valve foil can be attached by way of example along the attachment line to an intermediate body, either by gluing, welding or clamping, wherein the intermediate body in turn can be attached, for example, along an auxiliary attachment line directly or indirectly to the sleeve and/or to another intermediate body. The intermediate body can, for example, be formed by the frame.

The attachment line can have any desired shape. For example, it can be straight within the plane of the valve foil. The attachment line can particularly form a substantially straight line or partially surround the second opening.

When there is a positive pressure difference in the blocking direction of the check valve, the valve foil lies substantially flat against the sleeve (that is, apart from wrinkles) and thereby covers the second opening in a fluid-tight manner. In contrast, when there is a negative pressure difference, the valve foil is pushed away from the sleeve in the areas outside of the attachment line in such a manner that fluid can pass through the check valve in the direction of passage.

The valve foil preferably has at least one aperture which is arranged outside of the second opening when the second opening is completely covered. The aperture is therefore offset with respect to the second opening in such a manner that the aperture and the second opening do not overlap when covered. The valve foil then lies, when there is a positive pressure difference in the blocking direction of the check valve, substantially (that is, apart from wrinkles) flat against the sleeve, and is substantially parallel to the surface thereof. In this way, it covers the second opening in a fluid-tight manner.

In particular, the attachment line can run in a way such that the second opening and the aperture are enclosed together by the line. The accordingly reduced mobility achieves a higher operational reliability at a higher density. In particular, the valve foil can be forced by the body fluid into the second opening, through the sleeve attachment which is present around all sides of the second opening, only to a defined maximum depth. The depth depends both on the relative area of the surface of the valve foil bounded by the attachment line in relation to the area of the sleeve bounded by the attachment line (or, if present, the auxiliary attachment line), and on the elasticity of the valve foil and the sleeve. If the valve foil and/or the sleeve is under internal tensile stress due to the attachment, the maximum depth is also dependent on the internal tensile stress.

It is advantageous if the aperture of the valve foil has an area between 5 mm² and 500 mm². It is particularly advantageous if the aperture has an area between 10 mm² and 200 mm². It is also possible that the valve foil has a plurality of apertures, all of which lie outside of the second opening when the second opening is covered. In this case, the apertures of the valve foil together preferably includes a total area of between 5 mm² and 500 mm², and particularly preferably between 10 mm² and 200 mm² (wherein a single aperture of the plurality of apertures preferably has an area of between 0.25 mm² and 250 mm², and more preferably between 1 mm² and 20 mm²). This (total) aperture area enables, on the one hand, a high degree of tightness of the check valve, and on the other hand a low flow resistance for the fluid being conveyed. The attachment line can then, for example, run around all of the apertures together.

According to another aspect of the invention, the check valve can include an additional foil, which lies partially flat against the valve foil, and is attached (in a fluid-tight manner) to the sleeve, wherein the attachment line of the valve foil together with the attachment line of the additional foil surrounds the second opening. The second opening is then surrounded by the attachment lines of the valve foil and the additional foil. Such a foil valve can be designed as an outlet valve, for example in accordance with DE 35 25 165 A1 which is incorporated herein by reference.

Preferably, the sleeve is, at least partially, exactly or substantially radially symmetric or rotationally symmetric (infinitely radially symmetric) about a longitudinal axis—particularly beyond its ends, or at least its outer envelope ends—in particular with a cylindrical shape, and the second opening is arranged in a shell surface (surrounding the longitudinal axis), particularly a cylinder shell surface, of the sleeve. The smallest outer diameter corresponds to the cross-section along the longitudinal axis. The higher the degree of radial symmetry is, the smaller can be the smallest outer diameter, in an advantageous manner. It is expedient to arrange the third opening on the longitudinal axis.

The sleeve preferably includes (in addition to the line segment) a pump chamber segment. One or more connecting segments can be arranged between the line segment and the pump chamber segment. The pump chamber segment can be wholly or partially a subsegment of the line segment. Advantageously, the first opening of the sleeve is arranged in the region of one end of the line segment, and the second opening is arranged in the region of an opposite end of the line segment.

According to an aspect of the invention, the second opening is arranged in the region of the pump chamber segment of the sleeve. In this case, the pump chamber segment forms a part of the line segment such that the pump chamber is part of the line.

However, in an embodiment which is particularly suitable for use in the left heart, the second opening can also be arranged outside of the pump chamber segment in the line segment.

Typically, the sleeve has a greater inner diameter in the region of the pump chamber segment than in the region of the line segment.

The sleeve can be designed as one piece or several pieces. In one embodiment where there is a plurality of parts, the sleeve can advantageously be constructed of a part arranged outside of the frame, which furnishes the line segment, and a part arranged in the region of the frame, which provides the pump chamber segment. In one embodiment where the sleeve has a plurality of parts, the same can consist of different materials and be advantageously connected to each other in a fluid-tight manner. The sleeve or the parts thereof can also each be made of different materials.

In particular, there can be different materials making up at least two layers. For example, there can be a support layer which gives the sleeve or the part a predetermined mechanical rigidity and an envelope layer connected to the carrier layer to form the fluid-tight sleeve. If the frame is formed by the sleeve itself, the support layer can be formed by the frame.

The sleeve is preferably formed at least in the pump chamber segment by at least one sleeve foil. The sleeve foil can itself be multilayered. Preferably, the sleeve foil is at least locally (directly or indirectly—see above) connected to the frame. Due to its minimal thickness, the sleeve therefore occupies a minimal constructed space. This enables an even smaller outer diameter of the catheter (in the collapsed configuration of the frame) in the region of the pump chamber. The sleeve foil can be routed, for example as in a tissue, back and forth between the internal space and the external space through the struts of the frame.

The sleeve foil in this case can advantageously be deflected (relative to the frame) only to a lesser degree than the valve foil (with identical deflection force) and/or is subject to a greater internal tensile stress by the frame than the valve foil, particularly wherein the elasticity of the sleeve foil is less than that of the valve foil. This achieves, on the one hand, a good seal of the check valve, and on the other hand a low flow resistance for the conveyed fluid.

In all embodiments, the valve foil (and optionally the additional foil) can preferably be arranged inside the sleeve, in such a manner that the check valve functions as an intake valve. This configuration is particularly advantageous for use in the bloodstream in the right heart.

Alternatively, in all embodiments, the valve foil can be arranged outside of the sleeve in such a manner that the check valve functions as an outlet valve. The advantages of the invention are achieved in this way as well.

In an advantageous embodiment variant, the third opening of the sleeve is formed in such a manner that a drive, in particular a balloon of a balloon catheter, in particular an IABP, can be pushed through the third opening into the internal space of the sleeve to a predetermined target position relative to the sleeve. Preferably, the predetermined target position corresponds to the region of the pump chamber segment of the sleeve. The drive can be advantageously pushed through the third opening in such a manner that the same is closed off (in a fluid-tight manner, in particular at least relative to a maximum blood pressure). A catheter according to U.S. Pat. No. 5,460,607 A can be used as the drive, in the form of a displacement device, for example. U.S. Pat. No. 5,460,607 A is incorporated herein by reference.

The drive arranged in the internal space can be connected to an external energy source via a line leading through the third opening—in the case of a balloon catheter, for example, via an auxiliary fluid line to a pump console which can preferably fill and empty the balloon with an auxiliary fluid in a pulsing manner. Due to the displacing effect of the filled balloon, the pump console acts as a drive for the check foil valve according to the invention, and thus enables a directional transport of the body fluid. The drive can be adjusted with regard to the frequency of the filling operations of the balloon with auxiliary fluid and/or the volume of the auxiliary fluid per filling, for example. According to the invention, alternative embodiments of the drive can also be used—for example drives based on the principle of a piston pump or an impeller pump or drives based on the principle of a centrifugal pump.

According to an aspect of the invention, a balloon (a balloon catheter, in particular an IABP) is arranged in the internal volume of the sleeve, and a line for an auxiliary fluid is connected to the balloon for the (reversible) inflation of the balloon, wherein the line for the auxiliary fluid runs outward through the third opening of the sleeve. The third opening is then closed off in a fluid-tight manner by the line for the auxiliary fluid. As a result, the catheter is ready for use, without the additional steps of the subsequent introduction of a separate displacement device into the conveyed fluid, and the insertion of the balloon into the internal space of the line. The duration of treatment is reduced in this manner. In particular, the line for the auxiliary fluid can be connected, or is connected, to a pump (pump console) for the auxiliary fluid.

The frame is substantially tubular in design. Preferably, the frame is, at least partially, exactly or substantially radially symmetric or rotationally symmetric (infinitely radially symmetric) about a longitudinal axis—particularly beyond its ends, or at least its outer envelope ends—in particular with a cylindrical shape. In the catheter according to an aspect of the invention, the frame is preferably arranged at least partially along the region of the pump chamber segment of the sleeve. The frame is preferably deployable. The term “deployable” in the context of the invention means that the frame can be switched between two configurations with different internal volumes. Switching to the configuration with a greater internal volume can be called “deploying” and the other configuration can be called “folded.” In particular, the frame can be a deployable stent. The frame can be arranged in the internal space of the sleeve or on the outside around the sleeve. The frame can also be formed by the sleeve itself.

It can be advantageous if the frame forms (rigidly) at least a part of the sleeve, in particular the pump chamber segment, as well as the third opening, and particularly also the second opening. As a result, no additional material, which adds thereto, is needed for the sleeve.

The frame can advantageously include a composition which has a shape memory alloy, particularly nitinol, a shape memory polymer or a shape memory ceramic, or consists of the same. In particular, the ability to switch between the configurations can be reversible.

In a configuration with a greater internal volume, the frame tensions the sleeve in the region of its pump chamber segment to form a pump chamber. The fluid can be transported through the sleeve along the line implemented by the line segment either from the first opening to the second opening or vice-versa.

The transport direction depends on the orientation of the check valve arranged at the second opening.

The check valve can advantageously include a group of several second openings, wherein these openings of the respective group can be fully covered by the valve foil. This makes it possible to increase the fluid tightness and reduce the resistance to flow. The attachment line can then run around all of the second apertures of the group in question.

Particularly preferred are embodiments in which preferably a plurality of second openings is arranged, each with a check valve, in the shell surface of the substantially cylindrical sleeve, and in each of these is arranged a valve foil or a segment of the valve foil defined by lines of attachment, each with an aperture or a group of apertures (belonging to the respective check valve in question) to cover the respective second opening. In this way, the flow resistance of the catheter line can be reduced, with increased tightness.

Also advantageously, each check valve can include a group of several second openings, wherein the openings of the respective group of second openings can be fully covered by a valve foil or a segment (defined by an attachment line) of the valve foil. In this way, the flow resistance of the catheter line can be reduced, with increased tightness. The attachment line belonging to a group can then run, for example, around all of the respective second openings belonging to the group.

Preferably, a further valve, in particular a check valve, is arranged in the line for the body fluid or at the end of this line, which acts opposite to the at least one first valve. This improves the efficiency of the directional transport.

The line is preferably flexible, and particularly is a flexible tube.

The line for the body fluid has an elastic spiral at the end which is remote from the frame. In this way, the line end can be held at a predetermined position in the body, and particularly can be fixed in the blood vessel with a spacing therefrom on all sides.

Preferably, each second opening has an area of between 5 mm² and 500 mm².

It is particularly preferred that each second opening has an area between 10 mm² and 200 mm². In the event that the check valve includes a group of second openings, each group of second openings belonging to one check valve has a total area of between 5 mm² and 500 mm², and more preferably between 10 mm² and 200 mm² (wherein a single opening of the group of second openings advantageously has an area of between 0.25 mm² and 250 mm², and more advantageously between 1 mm² and 20 mm²). This (total) opening area enables, on the one hand, a high degree of tightness of the check valve, and on the other hand a low flow resistance for the conveyed fluid.

The first foil and/or the second foil can advantageously be made of at least one polymer, in particular polyurethane, in particular with a foil thickness of between 0.01 mm and 0.2 mm. This enables a first configuration of the catheter with a minimum constructed space.

### BRIEF DESCRIPTION OF THE DRAWINGS

The invention will be explained in more detail below with reference to drawings, wherein:

FIG. 1 shows a known line catheter with conventional check valves according to the prior art;

FIGS. 2A and 2B show a line catheter with multiple inlet check valves in the form of foil valves with separate first foils and integrated pump balloons, in two difference states;

FIG. 3 shows a line catheter with multiple inlet check valves in the form of foil valves with a common first foil;

FIG. 4 shows a line catheter with several inlet check valves in the form of foil valves with a common first foil, and for each of these, groups of several apertures and second openings;

FIG. 5 shows a line catheter with outlet check valves in the form of foil valves;

FIGS. 6A and 6B show a line catheter with multiple external outlet check valves in the form of foil valves, in two different states;

FIG. 7 shows a line catheter with alternative inlet foil valves;

FIG. 8 shows a second opening with an aperture offset with respect to the same, and with a possible attachment line;

FIG. 9 shows a line catheter with a plurality of inlet foil valves; and,

FIG. 10 shows a line catheter with a check outlet valve in the form of a foil valve in the region of the line segment outside the frame.

### DESCRIPTION OF THE PREFERRED EMBODIMENTS OF THE INVENTION

Matching parts in the different figures have the same reference numbers.

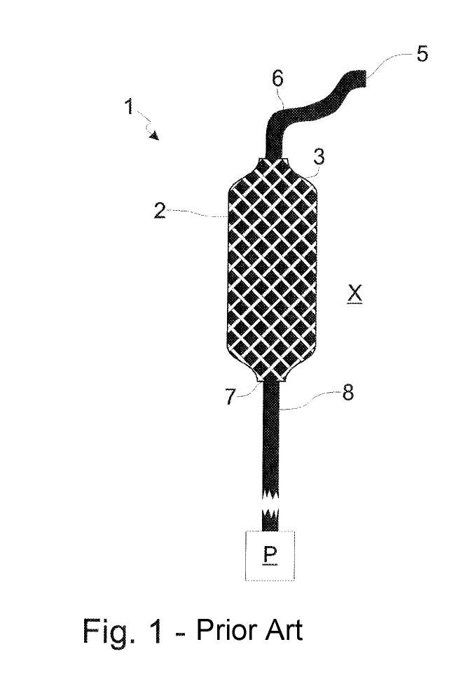

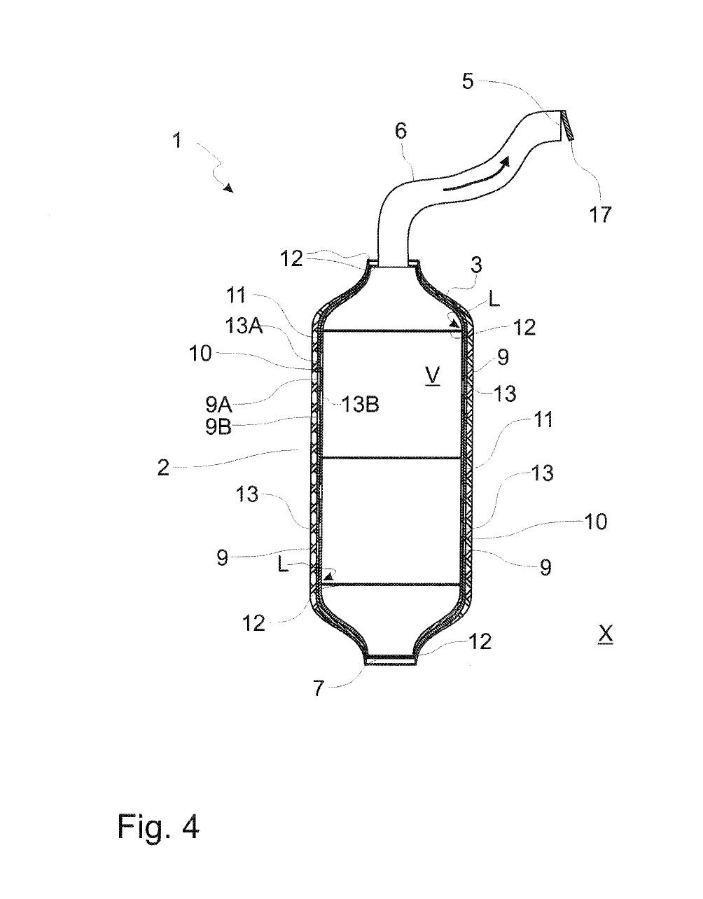

FIG. 1 shows a known catheter 1 in a schematic view from the outside. The catheter 1 includes a metal cage as a frame 2. In the interior of the metal cage, a sleeve 3 is arranged in such a manner that it has an internal space (not shown). A balloon (not shown) of a balloon catheter 4 is arranged in the internal space. The sleeve 3 includes, outside of the frame 2, a tube depicted as line 6 for the body fluid to be transported—in this case, by way of example, blood—at the end of which, remote from the frame 2, is arranged a first opening 5. The sleeve 3 has a plurality of second openings (not shown), wherein a check valve with mechanical flaps (not shown) is arranged as an inlet valve on each of the same. Regarding the second openings and check valves and their operation, reference is hereby made to U.S. Pat. Nos. 8,932,246 and 8,409,128, FIG. 4, which are incorporated herein by reference. Finally, the sleeve 3 has a third opening 7 through which the balloon catheter 4 is guided into the internal space of the sleeve 3. The balloon cannot be seen since the illustration is an external view. The sleeve 3 is closed off in a fluid-tight manner with respect to the local external space X. The fluid line 6 is open on the end thereof which faces away from the internal space, such that the fluid is transported from the internal space V through the line 6 and can exit at the end thereof. The second opening of the sleeve 3 is only permeable to fluid in the direction of the inlet, for example. In the opposite direction of flow, the second opening is closed off by the check valve. The third opening 7 of the sleeve 3 is then closed off in a fluid-tight manner by the line 8 of the balloon catheter. This auxiliary fluid line 8 is connected to an extracorporeal pump P which alternatingly pumps the auxiliary fluid into the balloon catheter and withdraws the same therefrom.

The balloon catheter 4 in this case functions as a drive for the line catheter 1 in the form of a positive displacement pump, specifically a diaphragm pump. The general operation thereof is described in U.S. Pat. Nos. 8,932,246 and 8,409,128 which are incorporated by reference.

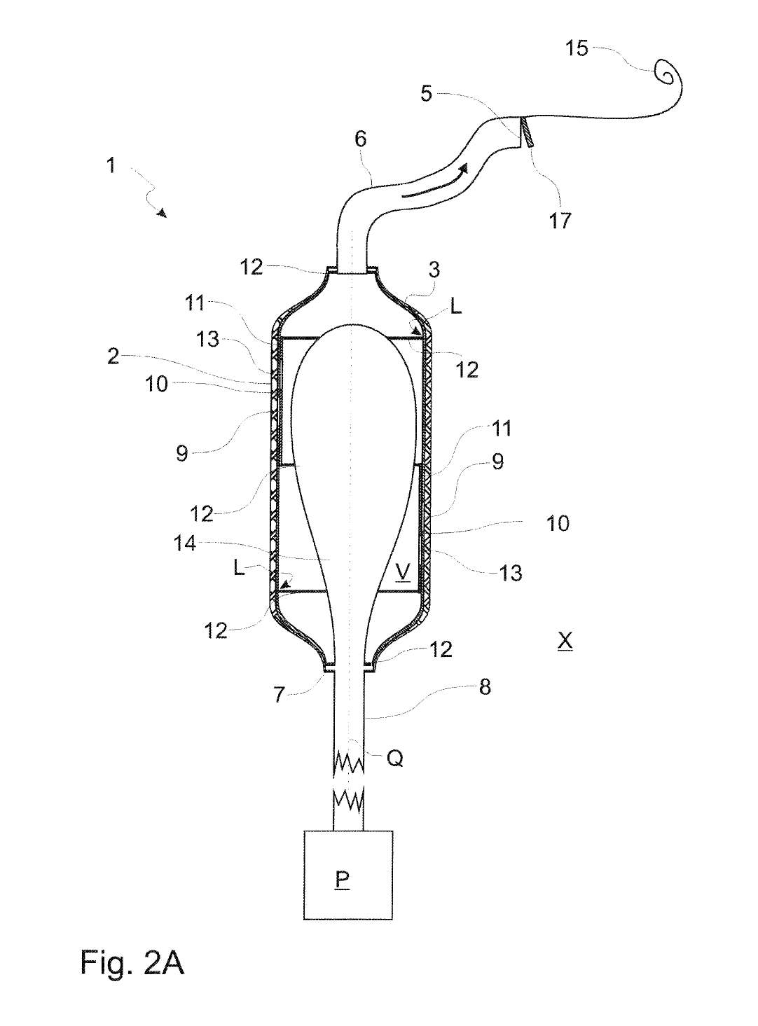

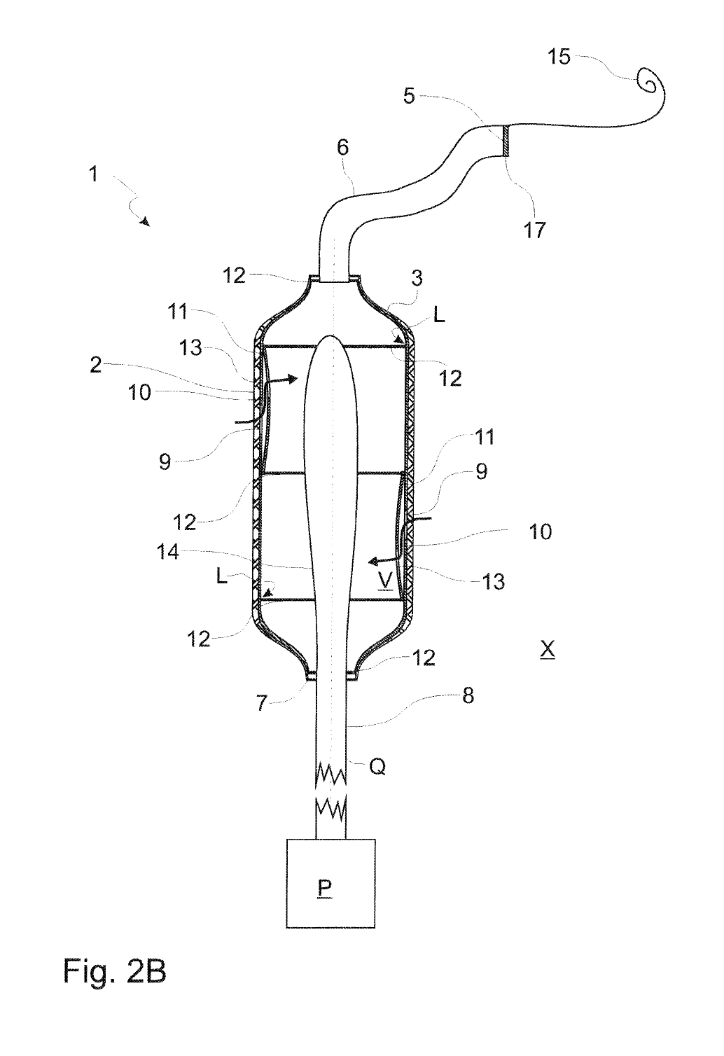

FIGS. 2A and 2B show in a schematic section view an improved catheter 1, the construction of which basically corresponds to that shown in FIG. 1. It can preferably be used as a right heart pump catheter. The check valves 10 which are arranged on the second openings 9 of the sleeve 3 are designed as foil valves. To this end, a valve foil 11 is attached to the sleeve 3 by clamping at each second opening 9 by rings 12, along the line L defined by the respective outer diameter of the respective ring. The cover 3, in the form of a sleeve foil which in this case clads the entire frame 2, by way of example, is arranged between the valve foils 11 and the frame 2. It is attached to the frame 2 by the rings 12, likewise by clamping. The balloon 14 of the balloon catheter 4 is arranged in the internal space V of the sleeve 3, the auxiliary fluid line 8 of which is guided out through the third opening 7 to the pump P.

Due to the clamping against the frame 2, the sleeve foil 3 is only able to be deflected a smaller distance from the frame 2, by a static pressure which is lower in the internal space V than in the external space X, than the valve foil 11, because it is subjected to a greater internal tensile stress by the frame 2 than the valve foil 11, and also has a lower elasticity than the valve foil 11. Each valve foil 11 has an aperture 13 which is offset with respect to the associated second opening 9 of the sleeve foil 3. When there is a static pressure which is lower in the internal space V than in the external space X, the body fluid being transported from the external space X through the openings 9 and the check valves 10 formed by the foils 11 and 3 can flow into the internal space V (the direction of passage, shown by arrows in FIG. 2B). When the opposite pressure condition prevails, for example during the inflation of the balloon 14 by the pump P by means of the auxiliary fluid, the valve foils 11 are pressed against the sleeve 3. Due to the offset between the second openings 9 and the apertures 13, the second openings 9 are covered by the valve foils 11 and thus closed off in a fluid-tight manner (the blocking direction, shown in FIG. 2A). Therefore, the body fluid being transported cannot flow from the internal space V through the opening 9 into the external space X. Rather, it must leave the internal space V through the line 6.

The catheter 1 can switch between two configurations in the segment in which the frame 2 is arranged, which differ in terms of the volume of the internal space V and in terms of the smallest outer diameter of the frame 2. The ability to switch is made possible due to the radially symmetric structure of the frame 2 about the longitudinal axis Q, and its being composed of a shape memory alloy, such as nitinol, as well as the flexible design of the check valves 10 and their arrangement in the shell surface of the essentially cylindrical frame. In the first configuration, the frame is folded such that it has an outer diameter of only 20 Fr at the thickest point. In the second configuration, it is unfolded such that the balloon 14 can be inflated. The frame 2 can be formed by a known, deployable stent, by way of example.

The foils 3 and 11 consist of polyurethane, for example, but can also be made of a different material, in particular another polymer. They are, by way of example, 0.1 mm thick, such that the foil valves 10 each have a thickness of less than 1 mm. All openings 5, 7, 9 of the sleeve 3 are, by way of example, circular with an opening area of, for example 5 mm², but can have any other shape and different sizes.

The same is true for the apertures 13 of the valve foils 11. The openings 5, 7, 9 and apertures 13 are produced, by way of example, by stamping, but also can be cut with a laser or produced in another manner.

A check valve 17 is arranged, as an outlet valve, at the first opening 5, which is arranged on the end of the fluid line 6 facing away from the internal space V, to improve the line efficiency; and an elastic spiral 15 is arranged, for the purpose of better fixing the line end, in a position with free space on all sides thereof from the vessel wall.

Each foil valve 10 opens and allows a fluid, such as blood, to flow through the outer opening 9 when the valve foil 11, which, apart from the rings 12, is not taut relative to the frame 2, which due to its relative rigidity makes the internal space V into a pump chamber, is drawn somewhat inward by the drive (suction or vacuum effect) of the inner balloon 14 which has just been emptied (in the systole, in the case of an application example of a pump catheter), as shown in FIG. 2B. Because of the briefly-formed channel between the outer opening 9, which is always open, and the inner aperture 13 which is temporarily drawn inward, the fluid can flow into the pump chamber V through the foil valve 10, which is normally operated in a pulsed manner, in a directional, time-controlled manner. In contrast, the foil valve 10 is closed rhythmically in the inflation phase of the balloon 14 (the diastole, in the application example of a pump catheter), as shown in FIG. 2A, when the inner catheter 4, for example an IABP catheter, driven by an external gas flow, is abruptly inflated, and the valve foil 11, due to the pressure increase in the pump chamber V, lies against the sleeve foil 3 cladding the pump chamber V. In this case, the staggered or offset openings 9 and apertures 13 of both foils 3, 11 of the foil valve 10 close, and the fluid, particularly blood, situated inside the pump chamber V, can be pumped directionally in a preferred direction (in the case of a pump catheter, in the distal direction along the line 6). The transport direction is shown by an arrow in the line 6, and/or by arrows in the check valves 10. As such, the foil valves 10, which can be arranged and formed in any arbitrary manner as regards their number and their form on the shell surface of a catheter used inside the body, play a decisive role in making it possible for fluids, preferably blood, to flow directionally in a pump catheter.

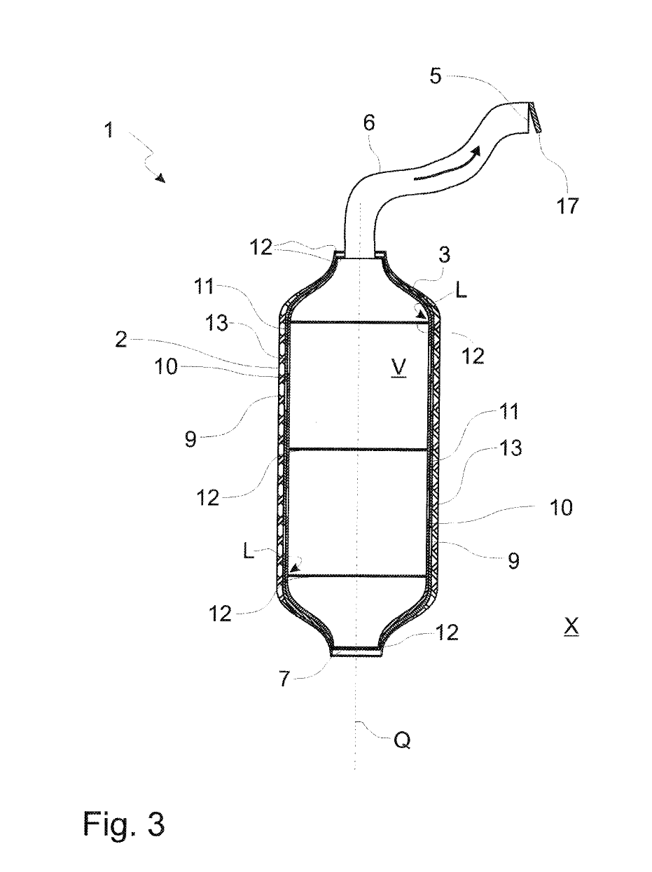

FIG. 3 shows a schematic segmental view of a line catheter which is modified compared to FIGS. 2A and 2B. In this case, the sleeve 3, which in turn includes a sleeve foil, is clad by a single valve foil 11 which is attached to the sleeve foil by rings 12. The respective segments of the sleeve foil 3 and valve foil 11 between the individual rings 12 differ in their ability to be deflected with respect to the frame 2 when there is low pressure in the internal space V. The segments of the valve foil 11 can be moved further inward than the segments of the sleeve foil 3 by the same force. This is achieved, by way of example, due to a difference elasticity or tension or geometry of the foils 3 and 11.

In order to realize the greater deflectability of the first foil 11 in an alternative manner, the first foil 11 can be clamped against the frame 2 with fewer rings 12 than the sleeve 3, by way of example.

In all embodiments, the rings 12 shown can be constructed of, for example, shape memory ceramic, shape memory metal, or as mechanical connection points produced by gluing, clamping or welding of the foil 11 and the sleeve 3. The rings 12 can also be part of the frame 2. Instead of separate rings 12, other geometries can be included—for example a single, continuous spiral. Instead of separate rings 12, there can be regularly or irregularly distributed—by way of example point-shaped—connection points (for example, a plurality of glued or welded points).

FIG. 4 shows a schematic segmental view of an embodiment which is modified compared to FIG. 3. Each check valve 10 in this case includes two second openings 9A and 9B in the sleeve 3, and two apertures 13A and 13B in the valve foil 11 which are offset thereto.

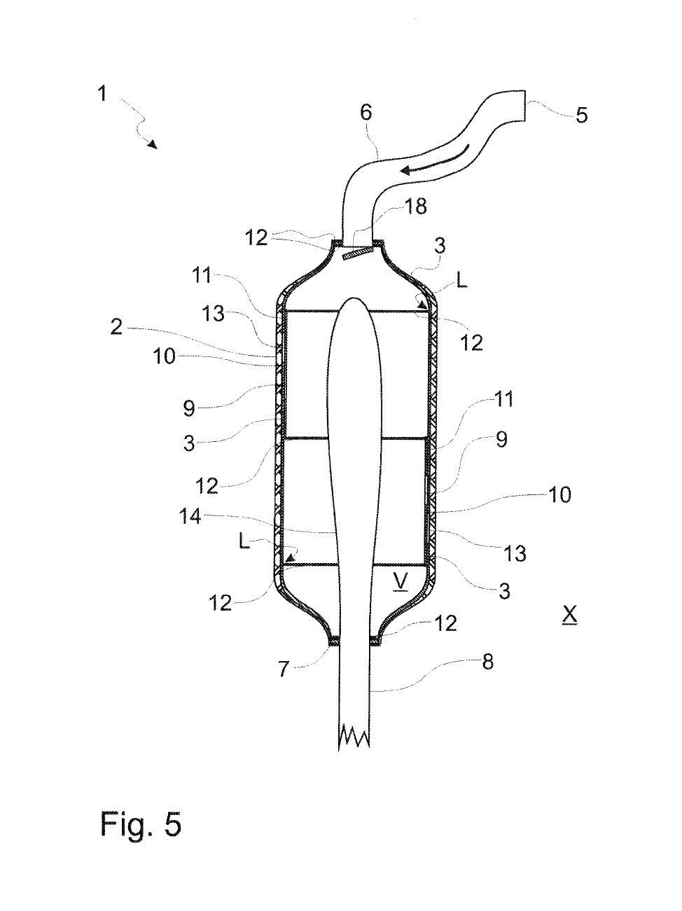

FIG. 5 again shows a schematic segmental view of a line catheter 1 which is modified compared to FIGS. 2A and 2B. It can preferably be used as a left heart pump catheter. In this case, the check valves 10 are formed as outlet foil valves. The valve foils 11 are arranged for this purpose between the sleeve 3 and the frame 2 and again connected to the sleeve at, for example, distributed points, or along an attachment line in the manner of a ring. Because of the additional deflectability of the valve foil 11, the same can, when there is an overpressure in the internal space V, yield into intermediate spaces of the nitinol stent which forms the frame 2, thereby enabling flow from the internal space V through the second openings 9 and the apertures 13 into the external space X. When there is underpressure in the internal space V, the valve foil 11 is drawn against the sleeve 3, thereby covering its second openings 9 in such a manner that the check valves 10 are locked. To support the conveyance efficiency, a check valve 18 is arranged as an inlet valve on the end of the fluid line 6 arranged on the frame 2.

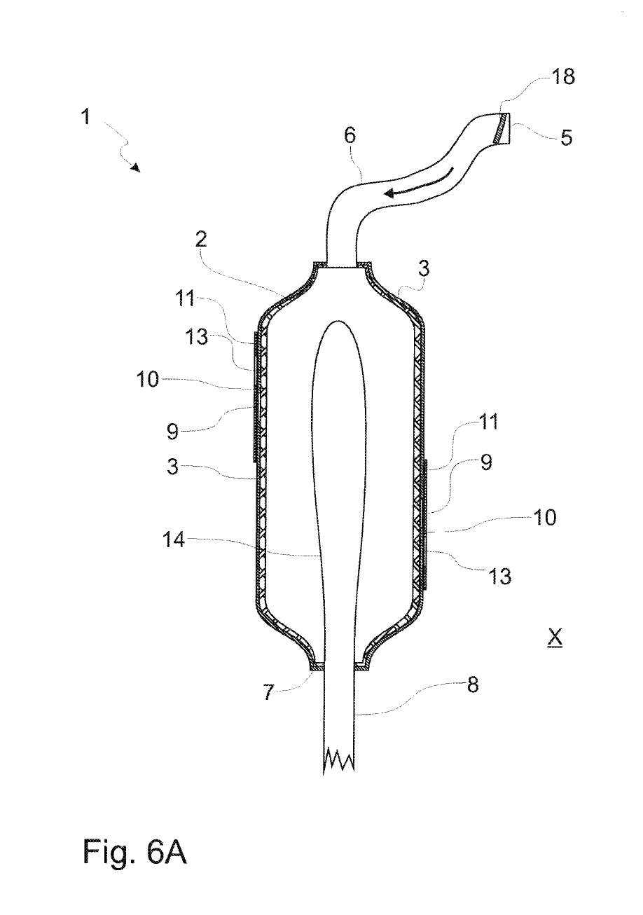

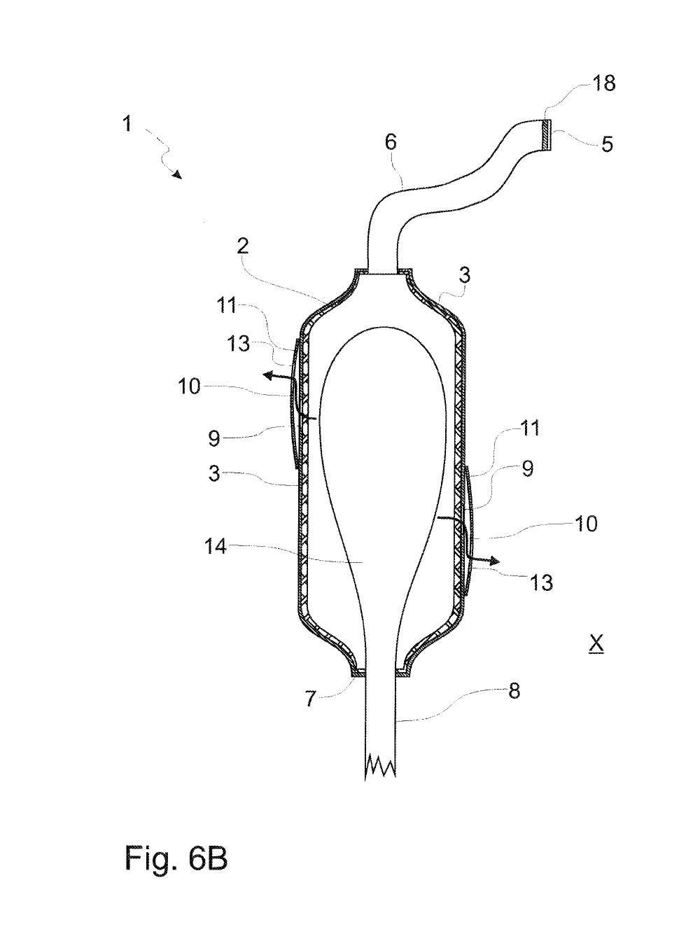

FIGS. 6A and 6B again show a schematic segmental view of a line catheter 1 which is modified compared to FIGS. 2A and 2B. The sleeve 3 in this case is arranged outside of the frame 2 and fixed to it, for example by gluing or welding. The first foil 11 is attached to the outside of the sleeve 3 to implement an outlet valve, for example by a joining method such as welding, soldering or gluing. Alternatively (not shown), it can be attached by clamping rings. In order to realize the opposite transport direction in an alternative embodiment (not shown), the first foil 11 would be arranged on the inside of the sleeve 3 to implement an inlet valve, for example inside the frame 2. The first foil 11 would then be attached on the frame 2 and thus only indirectly to the sleeve 3, or directly to the foil 3 through interstices of the frame 2.

FIG. 6B shows how the fluid during the filling of the balloon 14 is pressed from the internal space V through the outlet foil valves 10, as a result of the first foil 11 being lifted of the sleeve 3 by the fluid. FIG. 6A shows how during the emptying of the balloon 14, fluid is sucked through the line 6.

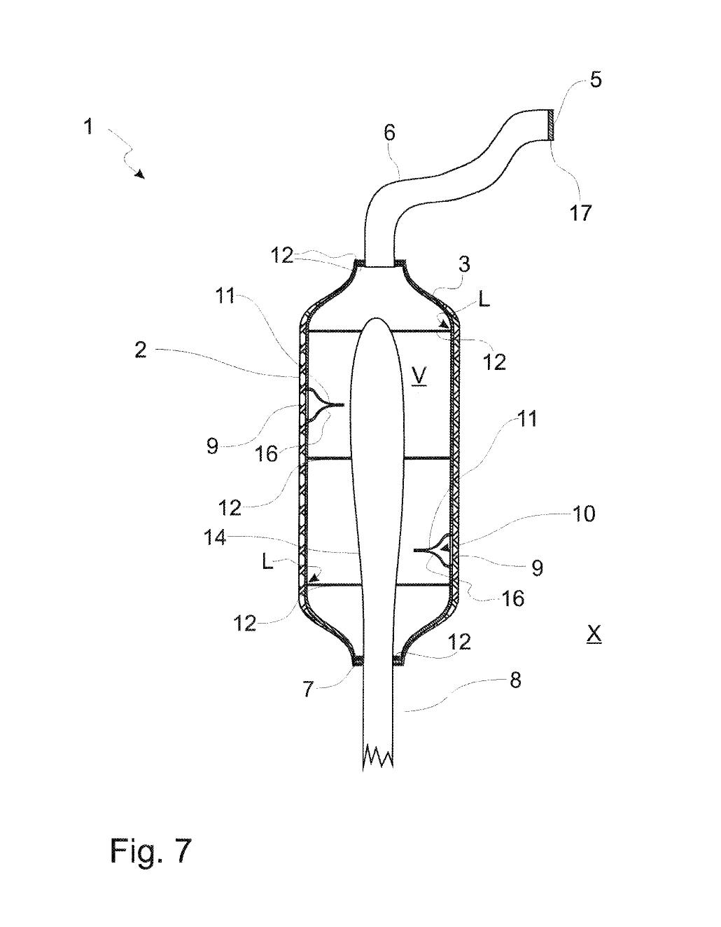

FIG. 7 shows an embodiment with an alternative shape of the foil check valves 10. In addition to a valve foil 11, the check valves 10 have an additional foil 16 which lies partially flat against the valve foil 11. Both foils are attached around the respective second opening 9 to the sleeve 3, which is also made of a polyurethane foil, by way of example. When there is an underpressure in the internal space V, the fluid can push the adjacent sheets 11 and 16 apart and thus flow through the same. When there is an overpressure in the internal space V, however, the two foils 11 and 16 are pressed against each other and are impermeable to fluid, such that no outward flow is possible.

Such a foil intake valve can be used as a check valve 10 on the end of the fluid line 6 arranged on the frame 2, for example in the embodiment according to FIG. 5. The same design of foil check valves 10 can also be used as an outlet valve, in particular in other embodiments—for example according to FIGS. 2A and 2B, at the first opening 5 on the end of the fluid line 6 remote from the frame 2.

In all embodiments, instead of clamping, a different kind of attachment can be used.

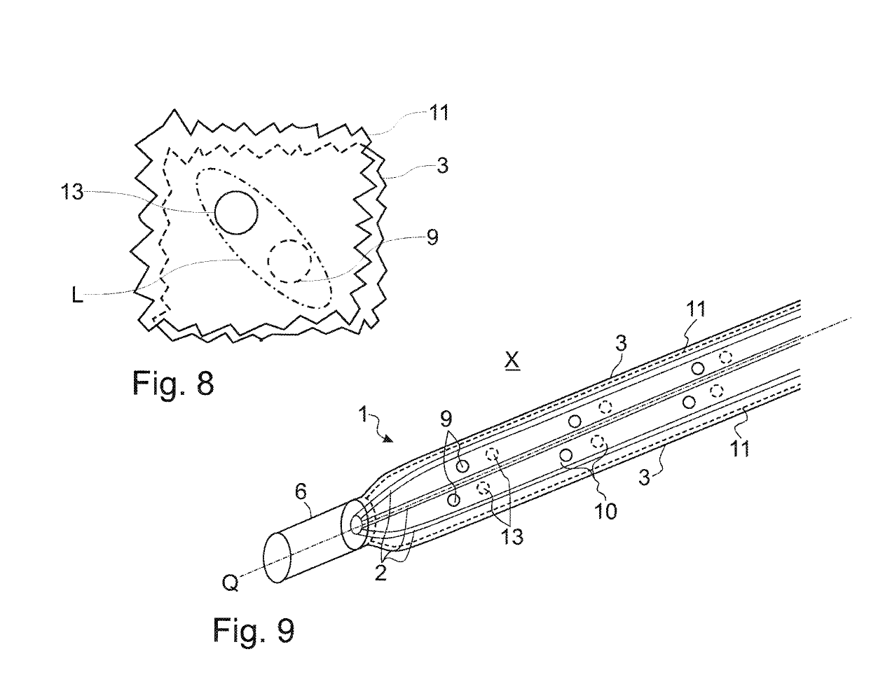

FIG. 8 shows a schematic segment of the sleeve 3 with a second opening 9 and the valve foil 11 arranged in front of the sleeve 3, with an aperture 13 offset with respect to the second opening. Around the opening 9 and the aperture 13, together, the valve foil 11 is attached to the sleeve 3 along the line L, for example by gluing or welding.

FIG. 9 shows an embodiment of a catheter 1 according to the invention, having a plurality of foil check valves 10, in a perspective view. The frame 2 is formed by a deployable laser-cut nitinol stent, wherein intermediate spaces are constructed between the individual longitudinal struts of the same. The sleeve 3 is formed by a flexible foil tube. The frame 2 is pushed into the sleeve foil tube 3.

The sleeve tube 3 encompasses the frame 2 tautly, at least in its deployed configuration—that is, with a predetermined pre-tension. The frame thus serves to stiffen the inner volume V formed by the sleeve, which constitutes a pump chamber in the embodiment of FIG. 9, wherein the balloon of a balloon catheter can be placed in said chamber. The sleeve 3 has a plurality of laser-cut or punched second openings 9. The valve foil 11 is likewise a single foil tube with a plurality of laser-cut or punched apertures 13. The valve foil tube 11 is arranged between the sleeve foil tube 3 and the frame 2. The foil of the valve foil tube 11 can be deflected by a given force (corresponding to an underpressure in the internal space V) further into the intermediate spaces of the frame 2 and into the pump chamber than the sleeve foil 3, as a result of its elasticity which is greater compared to the sleeve foil 3, such that the foil check valves 10 work as inlet valves. The second openings 9 and the apertures 13 are arranged in each case in a spiral around the longitudinal axis Q of the pump chamber. The foil tubes 3 and 11 are oriented with respect to each other such that the spirals of the apertures 13 lie outside of—that is, next to—the spiral of the openings 9, in such a manner that the apertures 13 and the openings 9 are offset relative to each other such that they do not overlap each other. Each opening 9 is therefore assigned to an aperture 13 such that both together form one foil valve 10. The two foil tubes 3, 11 are bonded to each other on their ends along the attachment line L by a glue which is suitable for gluing the foils being applied in a ring shape along the line L. In addition, the two foil tubes 3, 11 are connected to each other in intervals along the longitudinal axis Q. Two spiral adhesive sheets (not shown) are included for this purpose, running on both sides, offset and parallel to the offset spiral paths of the openings 9 and the apertures 13, thus bounding a valve chamber between the two foils, which winds along the longitudinal axis Q around the pump chamber in a spiral. The valve chambers communicate via the openings 9 with the external space X and via the apertures 13 with the internal space V. Their volume when the foil valves 10 are closed—that is, when the valve foil 11 presses against the sleeve 3, is minimal. In the open state—that is, when the valve foil which is limited in its mobility by the adhesive sheets is lifted from the sleeve 3 and deflected into the internal space V—it has a predetermined volume, the size of which depends inter alia on the given force with which the foil of the valve foil tube 11 is deflected into the pump chamber.

In an alternative embodiment which is similar to that of FIG. 9 (not shown), likewise having also a plurality of foil check valves 10, the frame 2 is again formed by a deployable nitinol stent. The sleeve 3 and the valve foil 11 are again each formed by a flexible foil tube. The frame 2 is pushed into the sleeve tube 3 similarly to the embodiment of FIG. 9. The sleeve tautly surrounds the frame 2, at least in its deployed configuration, such that the sleeve forms a pump chamber V stiffened by the frame 2. The valve foil tube 11 is again arranged between the sleeve foil tube 3 and the frame 2. In contrast to the embodiment of FIG. 9, the openings 9 and apertures 13 are arranged in a plurality of groups, each of six openings 9 and six apertures 13 about the longitudinal axis Q in a ring pattern, offset with respect to each other, such that they do not overlap.

Each opening 9 is assigned to an aperture 13 such that both together form one foil valve 10. In contrast to the embodiment of FIG. 9, the valve foil tube 11 has a greater diameter than the sleeve foil. Because of this allowance, the valve foil tube 11 pushed into the sleeve foil tube forms six pockets in the longitudinal direction Q. The valve foil 11 is adhesively bonded to the sleeve 3 along the six attachment lines L formed by the boundary lines of the pockets, such that six valve chambers are formed along the longitudinal axis Q between the attachment lines L. The valve chambers are arranged in relation to the frame 2 in such a manner that the longitudinal struts of the frame 2 run along the attachment lines L. In other words, the valve chambers are thus arranged in the intermediate spaces running longitudinally between the struts, wherein one second opening 9 of each group of second openings 9 and one aperture 13 of each group of apertures 13 is functionally assigned to each valve chamber. Each valve chamber therefore communicates via the openings 9 assigned to it with the external space X, and via the apertures 13 assigned to it with the internal space V. The volume of the valve chambers is minimal when the foil valves 10 are closed—that is, when the valve foil 11 presses against the sleeve 3. The valve foil 11 then lies substantially—that is, except for the folds that can potentially form due to the allowance—flat against the sleeve foil 3, such that the foil valves 10 are closed off in a fluid-tight manner. In the open state—that is, when the valve foil 11 is lifted by a given force (corresponding to an underpressure in the pump chamber V) off of the sleeve 3 and deflected into the internal space V, the valve chambers then have a predetermined volume, the magnitude of which, inter alia, depends on the force and the allowance.

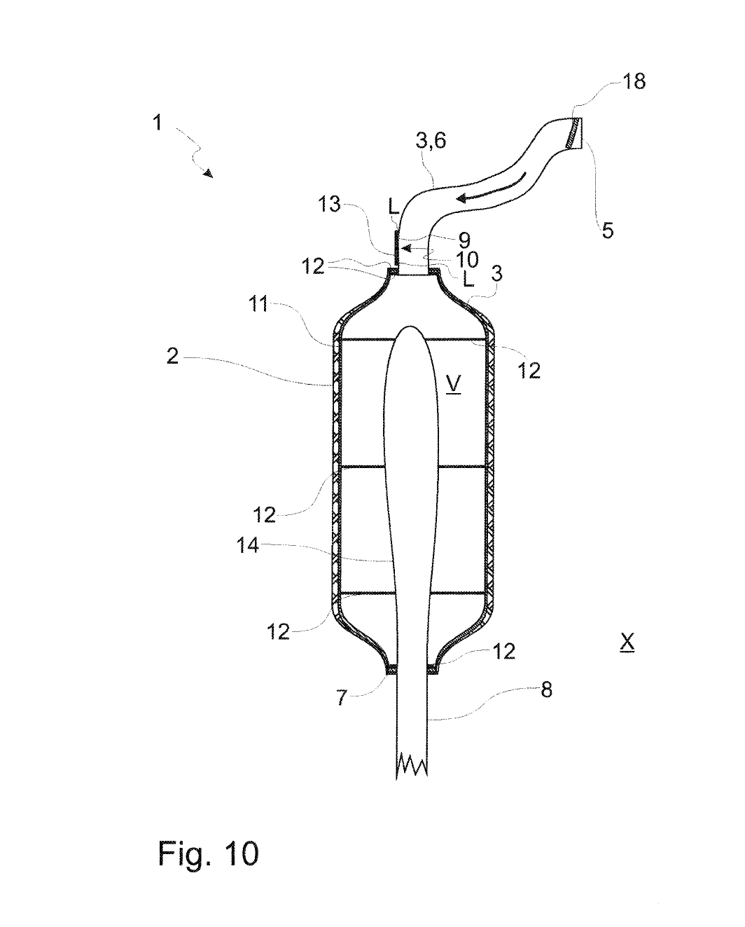

Finally, FIG. 10 shows a schematic segmental view of a line catheter 1 which is modified compared to FIGS. 2A and 2B. Here, the second opening 9 is arranged in the line 6. The pump segment of the internal space V has no opening to the external space X. A foil outlet valve 10 is implemented on the second opening 9 by a first foil 11 with an aperture 13. An inlet check valve 18 is arranged on the end of the line 6 facing away from the frame 2. If the balloon 14 creates an overpressure in the internal space V, the fluid is forced through the outlet valve foil 10 into the external space X. If the balloon 14 contracts, an underpressure is created in the internal space V such that fluid is drawn through the inlet valve 18 into the line and therefore the internal space V. In the next overpressure cycle, the fluid is then ejected through the outlet valve 10 and thereby conveyed along the line 6.

In all embodiments, a plurality of additional check valves 17 or 18 can be arranged at any point of the line 6, and in particular on the longitudinal axis thereof.

Alternatively or additionally, the additional valve 17/18 or the plurality of valves 17/18 can be arranged outside of the longitudinal axis of the line 6 in jacket of the line 6.

It is understood that the foregoing description is that of the preferred embodiments of the invention and that various changes and modifications may be made thereto without departing from the spirit and scope of the invention as defined in the appended claims.

### LIST OF REFERENCE NUMERALS

1 catheter
2 frame
3 sleeve
4 balloon catheter
5 first opening
6 line
7 third opening
8 auxiliary fluid line
9 second opening
10 check valve
11 valve foil
12 ring
13 aperture
14 balloon
15 spiral
16 additional foil
17 outlet check valve
18 inlet check valve
L line
V internal space
S segment
P pump
Q longitudinal axis
X external space

What is claimed is:

1. A catheter for directional conveyance of a fluid, the catheter comprising:
a sleeve having an internal space;
a frame;
said sleeve having at least a first opening, a second opening, and a third opening;
said sleeve, at least in a region between said first opening and said second opening, being configured as a conduit for conducting the fluid;
a check valve having a valve foil;
said check valve being arranged at said second opening; and,
said valve foil being at least partially attached to said sleeve so as to cause said second opening to be completely coverable by said valve foil.

2. The catheter of claim 1, further comprising:
said valve foil being connected to said sleeve along an attachment line; and,
said valve foil having at least one aperture which is arranged offset to said second opening when said second opening is completely covered.

3. The catheter of claim 2, wherein said second opening and said at least one aperture are surrounded by said attachment line.

4. The catheter of claim 2, wherein said at least one aperture has an area between 5 mm² and 500 mm².

5. The catheter of claim 2, wherein:
said valve foil of said check valve has a plurality of said apertures; and,
said plurality of apertures of said check valve conjointly define an area between 5 mm² and 500 mm².

6. The catheter of claim 1, wherein:
said catheter defines a longitudinal axis;
said sleeve includes a sleeve segment arranged in surrounding relationship to said longitudinal axis and said sleeve segment defines a lateral surface; and,
said second opening is arranged in said lateral surface.

7. The catheter of claim 6, wherein:
said sleeve having respective ends offset from said sleeve segment;
said sleeve segment is essentially radially symmetrical or rotationally symmetrical with respect to said longitudinal axis;
said lateral surface of said sleeve segment is a cylindrical lateral surface; and,
said third opening is arranged on said longitudinal axis.

8. The catheter of claim 7, wherein:
said respective ends of said sleeve are the outer respective ends thereof; and,
said sleeve segment is exactly radially symmetrical or rotationally symmetrical.

9. The catheter of claim 6, wherein said sleeve has a pump chamber segment and said second opening is arranged in said pump chamber segment of said sleeve.

10. The catheter of claim 6, wherein said conduit is a segment of said sleeve and said second opening is arranged in said conduit offset from said pump chamber.

11. The catheter of claim 9, further comprising:
at least one sleeve foil; and,
said sleeve being formed by said at least one sleeve foil at least in said pump chamber segment.

12. The catheter of claim 11, wherein:
said at least one sleeve foil is at least one of being deflected less far than said valve foil and being subjected by said frame to a greater internal tensile stress than said valve foil; and,
said sleeve foil has a lower elasticity than said valve foil.

13. The catheter of claim 1, wherein:
said valve foil is arranged inside said sleeve so as to cause said check valve to act as an inlet valve.

14. The catheter of claim 1, wherein:
said valve foil is arranged outside of said sleeve so as to cause said check valve to act as an outlet valve.

15. The catheter of claim 1, further comprising:
a balloon arranged in said internal space;
an ancillary conduit connected to said balloon and configured for conducting an ancillary fluid to said balloon for inflating the same; and,
said ancillary conduit passing through said third opening of said sleeve so as to run outside thereof.

16. The catheter of claim 15, further comprising a pump for said ancillary fluid; and, said pump being configured to be connected to or connectable with said ancillary conduit.

17. The catheter of claim 1, further comprising:
a plurality of said second openings;
a plurality of check valves assigned to corresponding ones of said second openings;
a plurality of said valve foils assigned to corresponding ones of said check valves; and,
each of said plurality of second openings being arranged on said sleeve with the check valves assigned thereto.

18. The catheter of claim 17, wherein respective segments of said valve foils are defined by corresponding attachment lines and said segments have respective apertures formed therein and are configured to cover corresponding ones of said second openings.

19. The catheter of claim 17, wherein respective segments of said valve foils are defined by corresponding attachment lines and said segments have respective groups of apertures formed therein and are configured to cover corresponding ones of said second openings.

20. The catheter of claim 1, wherein:
said check valve includes a group of several of said second openings; and,
each second opening of the group of second openings is entirely coverable by said valve foil.

21. The catheter of claim 1, wherein said fluid is a body fluid; and, said check valve is a first check valve; and, said catheter further comprises a second check valve arranged in said conduit for conducting said body fluid; and, said second check valve is configured to act opposite to said first check valve.

22. The catheter of claim 1, wherein said frame is composed of at least one of a composition of a shape memory alloy, a shape memory polymer and a shape memory ceramic.

23. The catheter of claim 22, wherein said shape memory alloy is nitinol.

24. The catheter of claim 17, wherein each of said second openings has an area between 5 mm² and 500 mm².

25. The catheter of claim 17, wherein said second openings are in groups of two corresponding to respective ones of said check valves and each of said groups has a total area of between 5 mm² and 500 mm².

26. The catheter of claim 1, wherein at least one of said valve foil and said sleeve foil are made of a polymer.

27. The catheter of claim 20, wherein said polymer is polyurethane.

28. The catheter of claim 26, wherein said polymer has a foil thickness of between 0.01 mm and 0.2 mm.
